(19) United States
(12) Patent Application Publication
(10) Pub. No.: US 2020/0129664 A1
(43) Pub. Date: Apr. 30, 2020

(54) CHAMBER FOR TRANSPLANTATION AND DEVICE FOR TRANSPLANTATION

(71) Applicant: FUJIFILM Corporation, Tokyo (JP)

(72) Inventors: Kuniyuki KAMINAGA, Ashigarakami-gun (JP); Ryuta TAKEGAMI, Ashigarakami-gun (JP); Kazuhiro HASEGAWA, Ashigarakami-gun (JP); Yusuke MOCHIZUKI, Ashigarakami-gun (JP)

(73) Assignee: FUJIFILM Corporation, Tokyo (JP)

(21) Appl. No.: 16/728,792

(22) Filed: Dec. 27, 2019

Related U.S. Application Data
(63) Continuation of application No. PCT/JP2018/024669, filed on Jun. 28, 2018.

(30) Foreign Application Priority Data
Jun. 29, 2017 (JP) 2017-127658

(51) Int. Cl. A61L 27/16; B01D 71/68; A61L 27/54; B01D 69/02; A61L 27/56; A61L 27/18

(52) U.S. Cl. CPC A61L 27/16; B01D 71/68; A61L 27/54; B01D 69/02; A61L 2300/43; A61L 27/18; B01D 2325/02; B01D 2325/04; A61L 27/56

(57) ABSTRACT

According to the present invention, there are provided a chamber for transplantation, including a membrane for immunoisolation at a boundary between an inside and an outside of the chamber for transplantation, in which the membrane for immunoisolation includes a porous membrane, and in the porous membrane, a maximum heat shrinkage ratio among heat shrinkage ratios in a membrane surface direction after immersion in water at 90° C. for 3 hours is 0.00% to 5.00%, and a difference between the maximum heat shrinkage ratio and a minimum heat shrinkage ratio among the heat shrinkage ratios in the membrane surface direction is 0.00% to 1.30%; and a device for transplantation including the chamber for transplantation enclosing a biological constituent therein. The chamber for transplantation of the present invention has a high durability and is capable of maintaining an enclosed biological constituent for a long period of time.

### CROSS-REFERENCE TO RELATED APPLICATIONS

This application is a continuation of PCT International Application No. PCT/JP2018/024669 filed on Jun. 28, 2018, which claims priority under 35 U.S.C § 119 (a) to Japanese Patent Application No. 2017-127658 filed on Jun. 29, 2017, the entire content of which is incorporated herein by reference.

### BACKGROUND OF THE INVENTION

### 1. Field of the Invention

The present invention relates to a chamber for transplantation which includes a membrane for immunoisolation, and a device for transplantation which includes the chamber for transplantation.

### 2. Description of the Related Art

Immunoisolation is one of methods for preventing immune reactions in a recipient during transplantation of biological constituents such as cells, tissues, or organs. A membrane for immunoisolation is a selectively permeable membrane which allows water, oxygen, glucose, or the like to permeate, and which, at the same time, performs immunoisolation by inhibiting permeation of immune cells and the like involved in an immune rejection. For example, while preventing an immune rejection, it is possible to achieve a purpose of transplantation by a device for transplantation utilizing a membrane for immunoisolation which allows physiologically active substances to permeate therethrough, for transplantation of cells secreting the physiologically active substances.

JP1996-507949A (JP-H08-507949A) discloses a chamber for transplantation formed of a polymeric porous membrane. It is disclosed that the chamber for transplantation includes a tube as a port means to provide selective access to the chamber.

### SUMMARY OF THE INVENTION

As disclosed in JP1996-507949A (JP-H08-507949A), in the chamber for transplantation in which the polymeric porous membrane is used as a membrane for immunoisolation, there are problems of a performance not being exhibited as designed or changes in performance over time.

An object of the present invention is to provide a chamber for transplantation having a high durability and capable of maintaining an enclosed biological constituent for a long period of time. Another object of the present invention is to provide a device for transplantation having a high durability and capable of maintaining an enclosed biological constituent for a long period of time.

The inventors of the present invention have conducted studies in an assumption that the deterioration in performance is derived from a change of pore diameter in the polymeric porous membrane. In order for the porous membrane to maintain a performance as a membrane for immunoisolation, it is considered that a physical structure of the porous membrane needs to be maintained. Particularly, because a pore diameter greatly affects permeation of substances, cells, or the like between an inside and an outside separated by the porous membrane, the pore diameter affects a performance of the chamber for transplantation whether it is larger or smaller than a designed value.

The inventors of the present invention have repeatedly conducted studies, have found that the above-mentioned objects can be achieved by production of a chamber for transplantation using a porous membrane in which a physical structure, especially a pore diameter, is difficult to change, and therefore have completed the present invention.

That is, the present invention provides the following <1> to <11>.

<1> A chamber for transplantation, comprising:

a membrane for immunoisolation at a boundary between an inside and an outside of the chamber for transplantation,

in which the membrane for immunoisolation includes a porous membrane, and

in the porous membrane, a maximum heat shrinkage ratio among heat shrinkage ratios in a membrane surface direction after immersion in water at 90° C. for 3 hours is 0.00% to 5.00%, and a difference between the maximum heat shrinkage ratio and a minimum heat shrinkage ratio among the heat shrinkage ratios in the membrane surface direction is 0.00% to 1.30%.

<2> The chamber for transplantation according to <1>, further comprising: a tube leading to the inside of the chamber for transplantation,

in which a part of the porous membrane and a part of the tube are fusion welded, and

the maximum heat shrinkage ratio is 0.05% or more.

<3> The chamber for transplantation according to <2>, in which the tube contains polyethylene, polyurethane, or polyvinyl chloride.

<4> The chamber for transplantation according to any one of <1> to <3>, in which the porous membrane contains a polymer.

<5> The chamber for transplantation according to any one of <1> to <4>, in which the membrane for immunoisolation is formed of a porous membrane containing a polymer.

<6> The chamber for transplantation according to any one of <1> to <5>, in which the porous membrane contains polysulfone or polyethersulfone.

<7> The chamber for transplantation according to any one of <1> to <6>, in which a thickness of the porous membrane is 25 μm to 100 μm.

<8> The chamber for transplantation according to any one of <1> to <7>, in which a bubble point diameter of the porous membrane is 0.02 μm to 25 μm.

<9> A device for transplantation, comprising the chamber for transplantation according to any one of <1> to <8> enclosing a biological constituent therein.

<10> The device for transplantation according to <9>, in which the biological constituent releases a physiologically active substance.

<11> The device for transplantation according to <10>, in which the physiologically active substance is insulin.

According to the present invention, it is possible to provide a chamber for transplantation having a high durability and capable of maintaining an enclosed biological constituent for a long period of time; and a device for transplantation having a high durability and capable of maintaining a biological constituent for a long period of time. In the chamber for transplantation of the present invention, defects such as peeling of a porous membrane are unlikely to occur even in a case of adopting a configuration in which a tube is provided as a port means for access.

### DESCRIPTION OF THE PREFERRED EMBODIMENTS

Hereinafter, the present invention will be described in detail.

In the present specification, “to” is used to refer to a meaning including numerical values denoted before and after “to” as a lower limit value and an upper limit value.

<<Chamber for Transplantation>>

A chamber for transplantation is a container for transplanting a biological constituent into a recipient. The chamber for transplantation can enclose the biological constituent therein.

The chamber for transplantation according to the embodiment of the present invention has a membrane for immunoisolation in a boundary (a boundary that separates the inside and the outside of the chamber for transplantation) between the inside and the outside thereof.

<Membrane for Immunoisolation>

In the present specification, a membrane for immunoisolation refers to a membrane used for immunoisolation.

Immunoisolation is one of a method for preventing an immune rejection by a recipient in a case of transplantation. Here, the immune rejection is a rejection by a recipient with respect to a biological constituent to be transplanted. A biological constituent is isolated from an immune rejection by a recipient due to immunoisolation. Examples of immune rejections include reactions based on cellular immune responses and reactions based on humoral immune responses.

The membrane for immunoisolation is a selectively permeable membrane that allows nutrients such as oxygen, water, and glucose to permeate therethrough, and inhibits permeation of immune cells and the like involved in an immune rejection. Examples of immune cells include macrophages, dendritic cells, neutrophils, eosinophils, basophils, natural killer cells, various T cells, B cells, and other lymphocytes.

Depending on the application, the membrane for immunoisolation preferably inhibits permeation of high-molecular-weight proteins such as immunoglobulins (IgM, IgG, and the like) and complements, and preferably allows a relatively low-molecular-weight physiologically active substances such as insulin to permeate therethrough.

The selective permeability of the membrane for immunoisolation may be adjusted according to the application. The membrane for immunoisolation may be a selectively permeable membrane which blocks a substance having a molecular weight such as 500 kDa or more, 100 kDa or more, 80 kDa or more, or 50 kDa or more. For example, it is preferable that the membrane for immunoisolation be capable of inhibiting permeation of the smallest IgG (molecular weight of about 160 kDa) among antibodies. In addition, the membrane for immunoisolation may be a selectively permeable membrane which blocks a substance having a diameter such as 500 nm or more, 100 nm or more, 50 nm or more, or 10 nm or more, as a sphere size.

The chamber for transplantation according to the embodiment of the present invention includes a membrane for immunoisolation at a boundary between an inside and an outside of the chamber for transplantation. In addition, the membrane for immunoisolation includes a porous membrane. The membrane for immunoisolation may be formed of only the porous membrane or may contain other layers such as a hydrogel membrane. The membrane for immunoisolation preferably has the porous membrane at least one surface thereof, and it is also preferable that the membrane for immunoisolation be formed of the porous membrane. A thickness of the membrane for immunoisolation is not particularly limited, but may be 10 μm to 500 is preferably 10 μm to 250 μm, and is more preferably 15 μm to 200 μm.

[Porous Membrane]

(Heat shrinkage Ratio of Porous Membrane)

In the porous membrane of the chamber for transplantation according to the embodiment of the present invention, the maximum heat shrinkage ratio among heat shrinkage ratios in a membrane surface direction is 0.00% to 5.00%, and a difference between the maximum heat shrinkage ratio and the minimum heat shrinkage ratio among the heat shrinkage ratios in the membrane surface direction is 0.00% to 1.30%. In a porous membrane which has a low heat shrinkage ratio and an equivalent heat shrinkage ratio in the membrane surface direction, a pore diameter is unlikely to be changed. The inventors of the present invention have found that, by using values within the above-mentioned range as specific values of heat shrinkage ratio, it is possible to provide a chamber for transplantation in which substance permeability is unlikely to change.

The maximum heat shrinkage ratio is preferably 2.00% or less and is more preferably 1.00% or less. In addition, a difference between the maximum heat shrinkage ratio and the minimum heat shrinkage ratio is preferably 0.50% or less and is more preferably 0.10% or less.

In the present specification, by immersing a porous membrane cut into a circular shape with a diameter of d mm in hot water at 90° C. for 3 hours, and then measuring the following A and B for the porous membrane in the wet state, the maximum heat shrinkage ratio, and a difference between the maximum heat shrinkage ratio and the minimum heat shrinkage ratio are obtained from the following formulas.

A: Length of shortest straight line among straight lines passing through center of circle immediately after cutting (mm)

B: Length of longest straight line among straight lines passing through center of circle immediately after cutting (mm)

(Maximum heat shrinkage ratio)=(d−A)/d×100%

(Minimum heat shrinkage ratio)=(d−B)/d×100%

(Heat shrinkage ratio difference)=(Maximum heat shrinkage ratio)−(Minimum heat shrinkage ratio)

(Structure of Porous Membrane)

The porous membrane is a membrane having a plurality of pores. Pores can be confirmed by, for example, captured images of a scanning electron microscope (SEM) or captured images of a transmission electron microscope (TEM) of a cross section of the membrane.

A thickness of the porous membrane is not particularly limited, but may be 10 μm to 500 μm, is preferably 10 μm to 250 μm, and is more preferably 15 μm to 200 μm.

A bubble point diameter of the porous membrane is preferably 0.02 μm to 25 μm, is more preferably 0.2 μm to 10 μm, and is even more preferably 0.5 μm to 5 μm. The bubble point is measured using a method of immersing the porous membrane in a liquid, increasing the air pressure from the lower side, and obtaining a pressure when bubbles are generated first from a pore with the maximum pore diameter of a neck portion. The pressure at this time is referred to as bubble point pressure, and the bubble point diameter can be obtained using a known formula based on the bubble point pressure. In a porous membrane not having pore diameter distribution in the thickness direction, the bubble point diameter usually corresponds to the maximum pore diameter of the porous membrane. In a porous membrane having pore diameter distribution in the thickness direction, the bubble point diameter corresponds to the maximum pore diameter of a compact portion described later.

The minimum pore diameter of the porous membrane is preferably 0.02 μm to 1.5 μm, and is more preferably 0.02 μm to 1.3 μm. The reason is that the minimum pore diameter of such a porous membrane can inhibit permeation of at least normal cells. The minimum pore diameter of the porous membrane can be measured by ASTM F316-80.

(Porous Membrane having Pore Diameter Distribution in Thickness Direction)

It is preferable that the porous membrane have pore diameter distribution in the thickness direction. In addition, the porous membrane preferably has a layered compact portion where a pore diameter is the smallest within the inside. Furthermore, it is preferable that a pore diameter continuously increase in the thickness direction from the compact portion toward at least one of the surfaces of the porous membrane. The pore diameter is determined by an average pore diameter of a parting line which will be described later.

The surface of the membrane means a main surface (a front surface or a back surface showing an area of the membrane), and does not mean a surface in the thickness direction of an end of the membrane. The surface of the porous membrane may be an interface with another layer. In the membrane for immunoisolation according to the embodiment of the present invention, it is preferable that the porous membrane have the same structure in an intra-membrane direction (a direction parallel to the membrane surface) with respect to pore diameters or pore diameter distribution (a difference in pore diameters in the thickness direction).

By using the porous membrane having pore diameter distribution, the life of the chamber for transplantation can be improved. The reason is that, by using a plurality of membranes having substantially different pore diameters, effects are obtained as though multistage filtration would be carried out, and therefore a deterioration in the membrane can be prevented.

A pore diameter may be measured from a photograph of a cross section of the membrane obtained by an electron microscope. The porous membrane can be cut with a microtome or the like, and it is possible to obtain a photograph of a cross section of the porous membrane as a section of a thin membrane which a cross section can be observed.

In the present specification, the comparison of pore diameters in the thickness direction of the membrane is performed by comparing pore diameters in 19 parting lines in a case where an SEM image of the cross section of the membrane is divided into 20 in the thickness direction of the membrane. 50 or more consecutive pores that intersect or are in contact with the parting line are selected, each of the pore diameters is measured, and an average value is calculated as an average pore diameter. Here, as the pore diameter, not a length of a portion where the selected pore intersects the parting line, but a diameter is used, the diameter being calculated using an area, which is obtained by calculating an area of pores calculated from an SEM image of the cross section of the membrane by image processing, as an area of a true circle. In this case, for a parting line in which pores are large and therefore only up to 50 pores can be selected, an average pore diameter is assumed to an average pore diameter obtained by measuring 50 pores by broadening the field of view of an SEM image for obtaining the cross section of the membrane. Pore diameters in the thickness direction of the membrane are compared by comparing the obtained average pore diameter for each parting line.

The layered compact portion having the smallest pore diameter refers to a layered portion of the porous membrane including the parting line where an average pore diameter becomes the smallest among parting lines in a photograph of the cross section of the membrane. The compact portion may include two or more parting lines. For example, in a case where two or more parting lines, which have an average pore diameter 1.1 times or less the minimum average pore diameter, are consecutive, the compact portion is assumed to include two or more consecutive parting lines. In the present specification, a thickness of the compact portion is a product of the number of parting lines included in the compact portion and one-twentieth of the thickness of the membrane.

In the porous membrane having pore diameter distribution in the thickness direction, the average pore diameter of the compact portion can be determined as the minimum average pore diameter of the porous membrane. It is preferable that the minimum pore diameter be measured by ASTM F316-80 after determining the compact portion.

It is preferable that the porous membrane having pore diameter distribution in the thickness direction have the compact portion within the membrane. The phrase “within the membrane” means that the compact portion is not in contact with the surface of the membrane. The phrase “having the compact portion within the membrane” means that the compact portion is not the closest section to any surface of the membrane. By using the porous membrane having a structure having the compact portion within the membrane, permeability of a substance intended to permeate is unlikely to lower compared to a case of using a porous membrane having the compact portion, which is in contact with the surface thereof Although not bound by any theory, it is perceived that protein adsorption is less likely to occur due to the presence of the compact portion within the membrane.

It is preferable that the compact portion be biased to one of the front surface side than a central portion in thickness of the porous membrane. Specifically, the compact portion is preferably located at a distance of less than half the thickness of the porous membrane from the surface of one of the porous membranes and it is even more preferably located within a distance of two-fifths. This distance may be determined from the photograph of the cross section of the membrane described above. In the present specification, the surface of the porous membrane closer to the compact portion is referred to as a “surface X.” In a case the porous membrane has the compact portion and the surface X, it is preferable in the chamber for transplantation that the surface X of the porous membrane be on the inside thereof. That is, it is preferable that the membrane for immunoisolation be disposed so that the compact portion of the porous membrane in the membrane for immunoisolation is closer to the inside of the chamber for transplantation. By setting the surface X in the inside of the chamber for transplantation, it is possible to make permeability of physiologically active substances higher.

In the porous membrane, it is preferable that a pore diameter continuously increase in the thickness direction from the compact portion toward at least one of the surfaces. In the porous membrane, the pore diameter may continuously increase in the thickness direction toward the surface X from the compact portion, the pore diameter may continuously increase in the thickness direction toward the surface opposite to the surface X from the compact portion, and the pore diameter may continuously increase in the thickness direction toward any surface of the porous membrane from the compact portion. Among them, it is preferable that the pore diameter continuously increase in the thickness direction toward at least the surface opposite to the surface X from the compact portion, and it is preferable that the pore diameter continuously increase in the thickness direction toward any surface of the porous membrane from the compact portion. The sentence “the pore diameter continuously increases in the thickness direction” means that a difference in average pore diameters between sections adjacent to each other in the thickness direction increases by 50% or less of a difference between maximum average pore diameters and minimum average pore diameters, preferably increases by 40% or less, and more preferably increases by 30% or less. The phrase “continuously increasing” essentially means that a pore diameter increases uniformly without decreasing, but a decreasing portion may occur accidentally. For example, in a case of combining two sections from the surface, in a case where an average value of a combination increases uniformly (uniformly decreases toward the compact portion from the surface), it can be determined that “the pore diameter continuously increases in the thickness direction toward the surface of the membrane from the compact portion.”.

The porous membrane having pore diameter distribution in the thickness direction can be realized by, for example, a manufacturing method to be described later. Particularly, it is preferable that the porous membrane having pore diameter distribution in the thickness direction be manufactured using a polymer selected from the group consisting of polysulfone and polyethersulfone.

In the porous membrane having pore diameter distribution in the thickness direction, an average pore diameter of a parting line having the maximum average pore diameter among parting lines can be determined as the maximum average pore diameter of the porous membrane. The maximum average pore diameter of the porous membrane having pore diameter distribution in the thickness direction is preferably 0.15 μm to 100 μm, is more preferably 1.0 μm to 50 μm, and is even more preferably 2.0 μm to 21 μm. It is preferable that the parting line where an average pore diameter becomes maximum be a parting line closest to any surface of the porous membrane.

In the porous membrane having pore diameter distribution in the thickness direction, a ratio of an average pore diameter (minimum average pore diameter) of the compact portion to the maximum average pore diameter (a ratio of a minimum average pore diameter to a maximum average pore diameter of the porous membrane, which is a value obtained by dividing the maximum average pore diameter by the minimum average pore diameter, an “anisotropy ratio” in the present specification) is preferably 3 or more, is more preferably 4 or more, and is even more preferably 5 or more. The reason is that an average pore diameter except for that of the compact portion increases to increase substance permeability of the porous membrane. In addition, the anisotropy ratio is preferably 25 or less, and is more preferably 20 or less. The reason is that effects, as though multistage filtration would be carried out, can be efficiently obtained within a range where an anisotropy ratio is 25 or less.

(Elemental Distribution of Porous Membrane)

Formulas (I) and (II) are preferably satisfied for at least one surface of the porous membrane.

B/A≤0.7 (I)

A≥0.015 (II)

In the formula, A represents a ratio of an N element (nitrogen atom) to a C element (carbon atom) on a surface of the membrane, and B represents a ratio of the N element to the C element at a depth of 30 nm from the same surface.

Formula (II) shows that a certain amount or more of N element is present on at least one surface of the porous membrane, and Formula (I) shows that an N element in the porous membrane is localized at a depth of less than 30 nm of the surface.

With the surface satisfying Formulas (I) and (II), a bioaffinity of the porous membrane, particularly, a bioaffinity of the surface side satisfying Formulas (I) and (II) becomes high.

In the porous membrane, either one of surfaces may satisfy Formulas (I) and (II), or both surfaces may satisfy Formulas (I) and (II), but it is preferable that both surfaces satisfy Formulas (I) and (II). In a case where either one of surfaces satisfies Formulas (I) and (II), the surface thereof may be in an inside or an outside of a chamber for transplantation to be described later, but the surface is preferably in the inside thereof In addition, in a case where only one of any surface satisfies Formulas (I) and (II) and the porous membrane has the above-mentioned surface X, a surface satisfying Formulas (I) and (II) is preferably the surface X.

In the present specification, a ratio (A value) of N element to C element on the membrane surface and a ratio (B value) of N element to C element at a depth of 30 nm from the surface are obtained by calculating using XPS measurement results. The XPS measurement is X-ray photoelectron spectroscopy, which is a method for irradiating a membrane surface with X-rays, measuring kinetic energy of photoelectrons emitted from the membrane surface, and analyzing a composition of elements constituting the membrane surface. Under conditions using a monochromated Al-Kα ray described in Examples, the A value is calculated from results at the start of sputtering, and the B value is calculated from time results, which are calculated that the ray is at 30 nm from the surface of the membrane measured from a sputtering rate.

B/A may be 0.02 or more, and is preferably 0.03 or more, and is more preferably 0.05 or more.

A is preferably 0.050 or more, and is more preferably 0.080 or more. In addition, A may be 0.20 or less, and is preferably 0.15 or less, and is more preferably 0.10 or less.

B may be 0.001 to 0.10, and is preferably 0.002 to 0.08, and is more preferably 0.003 to 0.07.

In a method for manufacturing the porous membrane which will be described later, the elemental distribution of the porous membrane, especially the distribution of an N element, can be controlled by a moisture concentration contained in the temperature-controlled humid air, a time to apply the temperature-controlled humid air, a temperature of a coagulation liquid, an immersion time, a temperature of a diethylene glycol bath for washing, an immersion time in the diethylene glycol bath for washing, a speed of a porous membrane manufacture line, and the like. The distribution of the N element can also be controlled by an amount of moisture contained in a stock solution for forming a membrane.

(Composition of Porous Membrane)

The porous membrane may contain a polymer. It is preferable that the porous membrane be essentially composed of a polymer.

The polymer forming the porous membrane is preferably biocompatible. Here, the term “biocompatible” means that the polymer has non-toxic and non-allergenic properties, but does not have properties such that the polymer is encapsulated in a living body.

The number average molecular weight (Mn) of the polymer is preferably 1,000 to 10,000,000, and is more preferably 5,000 to 1,000,000.

Examples of polymers include thermoplastic or thermosetting polymers. Specific examples of polymers include polysulfone, cellulose acylate such as cellulose acetate, nitrocellulose, sulfonated polysulfone, polyethersulfone, polyvinylidene fluoride, polyacrylonitrile, styrene-acrylonitrile copolymer, styrene-butadiene copolymer, saponified ethylene-vinyl acetate copolymer, polyvinyl alcohol, polycarbonate, an organosiloxane-polycarbonate copolymer, a polyester carbonate, an organopolysiloxane, a polyphenylene oxide, a polyamide, a polyimide, polyamideimide, polybenzimidazole, ethylene vinyl alcohol copolymer, polytetrafluoroethylene (PTFE), and the like. From the viewpoints of solubility, optical physical properties, electrical physical properties, strength, elasticity, and the like, polymers may be homopolymers, copolymers, polymer blends, or polymer alloys.

Among them, polysulfone, polyethersulfone, cellulose acylate, polyvinylidene fluoride, and polycarbonate are preferable, and polysulfone is more preferable.

In a case where polysulfone or polyethersulfone is used as the polymer, the porous membrane preferably further contains a hydrophilic polymer. Examples of hydrophilic polymers include polyvinylpyrrolidone, hydroxypropyl cellulose, and hydroxyethyl cellulose. Among these, polyvinylpyrrolidone is preferable. By combining polysulfone or polyethersulfone which are hydrophobic with the hydrophilic polymer, biocompatibility can be improved.

The porous membrane may contain other components other than the above-mentioned components as an additive.

Examples of additives include metal salts of inorganic acids such as sodium chloride, lithium chloride, sodium nitrate, potassium nitrate, sodium sulfate, and zinc chloride; metal salts of organic acids such as sodium acetate and sodium formate; other polymers such as polyethylene glycol; high polymer electrolytes such as sodium polystyrene sulfonate and polyvinyl benzyl trimethyl ammonium chloride; ionic surfactants such as sodium dioctyl sulfosuccinate and sodium alkyl sodium taurate; and the like. The additive may act as a swelling agent for a porous structure. As an additive, it is preferable to use a metal salt. The porous membrane containing polysulfone or polyethersulfone preferably contains lithium chloride.

The porous membrane is preferably a membrane formed from a single composition as a single layer, and preferably not has a laminated structure of a plurality of layers. By forming the porous membrane from one composition as a single layer, it is possible to manufacture the chamber for transplantation at low costs by a simple procedure.

(Method for Manufacturing Porous Membrane)

As a method for manufacturing a porous membrane, a flow-casting method is preferable. This is because a porous membrane which has a low heat shrinkage ratio and an equivalent heat shrinkage ratio in the membrane surface direction can be easily produced.

In the flow-casting method, the stock solution for forming a membrane containing a polymer is flow-cast on a support. By selecting an additive or a solvent which are contained in the stock solution for forming a membrane with the polymer, a desired porosity can be given to the manufactured membrane and the pore diameter can be adjusted.

As the support, a plastic film or a glass plate may be used. Examples of materials of the plastic film include polyester such as polyethylene terephthalate (PET), polycarbonate, acrylic resin, epoxy resin, polyurethane, polyamide, polyolefin, a cellulose derivative, silicone, and the like. As the support, a glass plate or PET is preferable, and PET is more preferable.

The stock solution for forming a membrane may contain a solvent. A solvent having high solubility of the polymer to be used (hereinafter referred to as “favorable solvent”) may be used depending on a polymer to be used. In a case of using a coagulation liquid described later in the manufacturing of the porous membrane having pore diameter distribution, a favorable solvent is a solvent quickly substituted with the coagulation liquid in a case where the membrane is immersed in the coagulation liquid. Examples of solvents include N-methyl-2-pyrrolidone, dioxane, tetrahydrofuran, dimethylformamide, dimethylacetamide, or a mixed solvent thereof in a case where the polymer is polysulfone and the like; dioxane, N-methyl-2-pyrrolidone, dimethylformamide, dimethylacetamide, dimethylsulfoxide, or a mixed solvent thereof in a case where the polymer is polyacrylonitrile and the like; dimethylformamide, dimethylacetamide, or a mixed solvent thereof in a case where the polymer is polyamide and the like; acetone, dioxane, tetrahydrofuran, N-methyl-2-pyrrolidone, or a mixed solvent thereof in a case where the polymer is cellulose acetate and the like. Among them, N-methyl-2-pyrrolidone is preferably used.

In addition to a favorable solvent, the stock solution for forming a membrane preferably use a solvent (hereinafter referred to as “non-solvent”) in which the solubility of the polymer is low but is compatible with the solvent of the polymer. Examples of non-solvents include water, cellosolves, methanol, ethanol, propanol, acetone, tetrahydrofuran, polyethylene glycol, glycerin, and the like. Among these, it is preferable to use water.

A concentration of the polymer as the stock solution for forming a membrane may be 5 mass % to 35 mass %, is preferably 10 mass % to 30 mass %. By setting the concentration thereof to 35 mass % or less, sufficient permeability (for example, water permeability) can be imparted to the obtained porous membrane. By setting the concentration thereof to 5 mass % or more, the formation of a porous membrane which selectively allows substances to permeate can be secured. An amount of additive to be added is not particularly limited as long as the homogeneity of the stock solution for forming a membrane is not lost by the addition, but is 0.5% by volume to 10% by volume respect to a general solvent. In a case where the stock solution for forming a membrane contains a non-solvent and a favorable solvent, a ratio of the non-solvent to the favorable solvent is not particularly limited as long as a mixed solution can be maintained in a homogeneous state, but is preferably 1.0 mass % to 50 mass %, is more preferably 2.0 mass % to 30 mass %, and is even more preferably 3.0 mass % to 10 mass %.

It is possible to produce a porous membrane having the above-mentioned pore diameter distribution by adjusting the type and amount of a solvent used in a stock solution for forming a membrane, and a drying method after flow-casting. Manufacture of the porous membrane having pore diameter distribution can be carried out by a method including, for example, the following (1) to (4) in this order.

(1) A stock solution for forming a membrane, which contains a polymer, if necessary an additive and, if necessary a solvent, is flow-cast on a support while being in a dissolved state.

(2) The surface of the flow-cast liquid membrane is exposed to temperature-controlled humid air.

(3) The membrane obtained after being exposed to temperature-controlled humid air is immersed in a coagulation liquid.

(4) A support is peeled off if necessary.

A temperature of temperature-controlled humid air may be 4° C. to 60° C., and is preferably 10° C. to 40° C. A relative humidity of the temperature-controlled humid air may be 20% to 95% and is preferably 30% to 90%. The temperature-controlled humid air may be applied at a wind speed of 0.1 m/s to 10 m/s for 0.1 seconds to 30 seconds, preferably 1 second to 10 seconds.

In addition, an average pore diameter and position of the compact portion can also be controlled by a moisture concentration contained in the temperature-controlled humid air and a time of applying the temperature-controlled humid air. An average pore diameter of the compact portion can also be controlled by an amount of moisture contained in a stock solution for forming a membrane.

By applying the temperature-controlled humid air to the surface of the liquid membrane as described above, it is possible to cause coacervation from the surface of the liquid membrane toward the inside of the liquid membrane by controlling evaporation of a solvent. By immersing the membrane in a coagulation liquid containing a solvent having low solubility of the polymer but compatible with the solvent of the polymer in this state, the above-mentioned coacervation phase is fixed as fine pores, and pores other than the fine pores can also be formed.

A temperature of the coagulation liquid may be −10° C. to 80° C. in a process of immersing the membrane in the coagulation liquid. By changing a temperature during this period, it is possible to control a size of a pore diameter up to a support surface side by adjusting a time from the formation of the coacervation phase on the support surface side to the solidification from the compact portion. In a case where a temperature of the coagulation liquid is raised, the formation of the coacervation phase becomes faster and a time for solidification becomes longer, and therefore the pore diameter toward the support surface side tends to become large. On the other hand, in a case where a temperature of the coagulation liquid is lowered, the formation of the coacervation phase becomes slower and a time for solidification becomes shorter, and therefore the pore diameter toward the support surface side is unlikely to become large.

The porous membrane having pore diameter distribution is preferably manufactured using a stock solution for forming a membrane containing a polymer selected from a group consisting of polysulfone and polyethersulfone, and more preferably manufactured using a stock solution for forming a membrane containing a polymer selected from the group consisting of polysulfone and polyethersulfone, and polyvinylpyrrolidone. In the stock solution for forming a membrane to manufacture the porous membrane, polyvinylpyrrolidone is preferably contained by an amount of 50 mass % to 120 mass %, and more preferably by an amount of 80 mass % to 110 mass %, with respect to a total mass of polysulfone and polyethersulfone. Furthermore, in a case where the stock solution for forming a membrane contains lithium chloride as an additive, lithium chloride is preferably contained by an amount of 5 mass % to 20 mass %, and more preferably by 10 mass % to 15 mass %, with respect to the total mass of polysulfone and polyethersulfone.

As the coagulation liquid, it is preferable to use a solvent having a low solubility of the polymer used. Examples of such solvents include water, alcohols such as methanol, ethanol, and butanol; glycols such as ethylene glycol and diethylene glycol; aliphatic hydrocarbons such as ether, n-hexane, and n-heptane; glycerol such as glycerin; and the like. Examples of preferred coagulation liquids include water, alcohols, or a mixture of two or more of these. Among these, it is preferable to use water.

After immersion in the coagulation liquid, it is also preferable to perform washing with a solvent different from the coagulation liquid that has been used. Washing can be carried out by immersing in a solvent. Diethylene glycol is preferable as a washing solvent. Distribution of an N element in the porous membrane can be adjusted by adjusting either or both of a temperature and an immersion time of diethylene glycol in which a film is immersed by using diethylene glycol as a washing solvent. In particular, in a case where polyvinylpyrrolidone is used as the stock solution for forming a membrane of the porous membrane, a residual amount of polyvinylpyrrolidone on the membrane can be controlled. After washing with diethylene glycol, furthermore, the membrane may be washed with water.

Regarding a method for manufacturing the porous membrane having pore diameter distribution, reference can be made to JP1992-349927A (JP-H04-349927A), JP1992-068966B (JP-H04-068966B), JP1992-351645A (JP-H04-351645A), JP2010-235808A, and the like.

[Other Layers]

The membrane for immunoisolation may contain layers other than the porous membrane.

Examples of other layers include a hydrogel membrane. As a hydrogel membrane, a biocompatible hydrogel membrane is preferable. Examples thereof include an alginic acid gel membrane, an agarose gel membrane, a polyisopropyl acrylamide membrane, a membrane containing cellulose, a membrane containing a cellulose derivative (for example, methyl cellulose), a polyvinyl alcohol membrane, or the like. The hydrogel membrane is preferably an alginic acid gel membrane. Specific examples of alginic acid gel membranes include a polyion complex membrane of alginic acid-poly-L-lysine-alginic acid.

<Structure and the Like of Chamber for Transplantation>

The membrane for immunoisolation is disposed on at least a part of the surface forming a boundary between the inside and the outside of the chamber for transplantation. By disposing in such a manner, it is possible to protect the biological constituent enclosed in the chamber for transplantation from immune cells and the like present outside, and to introduce nutrients such as water, oxygen, and glucose into the inside of the chamber for transplantation from the outside.

The membrane for immunoisolation may be disposed on the entire surface of a boundary between the inside and the outside of the chamber for transplantation, and may be disposed a part of the surface corresponding to an area of, for example, 1% to 99%, 5% to 90%, 10% to 80%, 20% to 70% %, 30% to 60%, 40% to 50%, or the like with respect to the entire area. The membrane for immunoisolation is preferably disposed on substantially the entire surface of the boundary between the inside and the outside of the chamber for transplantation. A surface on which the membrane for immunoisolation is disposed may be one continuous portion or may be divided into two or more portions.

In a case where the membrane for immunoisolation is not disposed on the entire surface of the boundary between the inside and the outside of the chamber for transplantation, it is sufficient that remaining surface is formed of a material such as an impermeable membrane not allowing permeation of nutrients such as oxygen, water, and glucose, in addition to cells and the like.

The chamber for transplantation may have a joint portion at which the membranes for immunoisolation face each other to be joined. A portion of the membrane for immunoisolation that is being joined is not particularly limited, but is preferably an end portion of the membrane for immunoisolation. In particular, it is preferable that end portions be joined to each other. In the present specification, in a case where the term “end portion” is used regarding the membrane, it means a peripheral portion or a part thereof having a constant width which is substantially in contact with the side surface (edge) of the membrane thickness. It is preferable that all of outer peripheries except an injection port and the like to be described later be joined to each other between the membranes for immunoisolation. For example, the chamber for transplantation preferably has a configuration in which two membranes for immunoisolation face each other and outer peripheries thereof are joined, or a configuration in which one membrane for immunoisolation having a line symmetric structure is folded into two and facing outer peripheries are joined.

Joining can be performed by adhesion using an adhesive, fusion welding, and the like. For example, adhering can be performed using a curable adhesive. Examples of adhesives include known adhesives such as epoxy-based adhesives, silicone-based adhesives, acrylic-based adhesives, and urethane-based adhesives.

In addition, the porous membranes may be joined to each other by sandwiching a thermoplastic resin therebetween and heating the sandwiched portion. In this case, a resin having a melting point lower than that of the polymer forming the porous membrane is preferably used as the thermoplastic resin. Specific examples of thermoplastic resins include polyethylene, polypropylene, polyurethane, polyvinyl chloride, polytetrafluoroethylene, polyethylene terephthalate, and polycarbonate. Among them, polyethylene, polypropylene, polyurethane, polyvinyl chloride, and polytetrafluoroethylene are preferable, and polyethylene, polyurethane, and polyvinyl chloride are more preferable.

Furthermore, the porous membranes in the membrane for immunoisolation may be fusion welded to each other in a state of coming in direct contact with each other by not sandwiching another material therebetween. By such fusion welding, it is possible to obtain a chamber for transplantation without having a problem derived from a resin sandwiched between the porous membranes or the like. In a case of using porous membranes which contain a polymer selected from the group consisting of polysulfone and polyethersulfone, the porous membranes can be fusion welded to each other to be integrated by heating at a temperature of glass transition temperature or higher of the polymer and lower than a melting point of the polymer. Specifically, regarding the heating for the fusion welding, it is sufficient for a temperature to be 190° C. or higher and lower than 340° C., and a temperature is preferably 230° C. or higher and lower than 340° C.

A shape of the chamber for transplantation is not limited, and may be a shape such as a pouched-like shape, a bag shape, a tube shape, a microcapsule shape, or a drum shape. For example, a drum-shaped chamber for transplantation can be formed by joining the membrane for immunoisolation to the top and bottom of a silicone ring. A shape of the chamber for transplantation is preferably a shape capable of preventing movement within a recipient in a case where the chamber for transplantation is used as a device for transplantation to be described later. Specific examples of shapes of the chamber for transplantation include a cylindrical shape, a disk-like shape, a rectangular shape, an egg shape, a star shape, a circular shape, and the like. The chamber for transplantation may be in a form of a sheet, a strand, a spiral, or the like. The chamber for transplantation may be a chamber for transplantation which encloses the biological constituent and becomes the above-described shape only in a case where the chamber for transplantation used as a device for transplantation to be described later.

The chamber for transplantation may contain a biocompatible plastic or the like for maintaining the shape and strength as a container. For example, the boundary between the inside and the outside of the chamber for transplantation may be formed from a porous membrane and a biocompatible plastic. In addition, in the chamber for transplantation of which the porous membrane is disposed on the entire surface of the boundary between the inside and the outside, a biocompatible plastic having a net-like structure may be further disposed on the outside of the boundary between the inside and the outside, from the viewpoint of strength.

<Injection Port>

The chamber for transplantation preferably includes an injection port or the like for injecting the biological constituent or the like into the chamber for transplantation. As the injection port, a tube leading to the inside of the chamber for transplantation may be provided.

The tube may contain a thermoplastic resin, for example. The thermoplastic resin preferably has a melting point which is lower than that of the polymer material of the porous membrane.

Specific examples of thermoplastic resins used in the tube include polyethylene, polypropylene, polyurethane, polyvinyl chloride, polytetrafluoroethylene, polyethylene terephthalate, polycarbonate, and the like. Among them, polyethylene, polypropylene, polyurethane, polyvinyl chloride, and polytetrafluoroethylene are preferable, and polyethylene, polyurethane, and polyvinyl chloride are particularly preferable.

For example, the tube is sandwiched between the membranes for immunoisolation in a manner of coming into contact with a part of the porous membrane, and thereby joining with the part thereof. Joining can be performed by fusion welding, adhesion using an adhesive, and the like. Among them, it is preferable to perform fusion welding. The fusion welding may be heat fusion welding. In a case of using a porous membrane which has the maximum heat shrinkage ratio of 5.00% or less and has an equivalent heat shrinkage ratio in the membrane surface direction, the membrane does not easily shrink during such heat fusion welding, and therefore peeling or the like at the joint portion hardly occurs.

In a case of performing fusion welding, the tube preferably contains a thermoplastic resin having a melting point which is lower than that of the polymer material of the porous membrane. The reason is that, in a case of performing fusion welding between the porous membrane and a tube containing a thermoplastic resin having a melting point which is lower than that of the polymer material of the porous membrane, the tube material is considered to be first melted at the time of heating so that the melted tube material can get into the pores of the porous membrane. In this case, it is particularly preferable to use a porous membrane having the maximum heat shrinkage ratio of 0.05% or more. This is because the porous membrane shrinks slightly, a portion infiltrates the pores is tightened, and the portion infiltrated the pores can be prevented from falling off from the pores. Since the portion infiltrated the pores and the tube are integrated, the porous membrane and the tube are difficult to be peeled off as a whole.

In a case of performing adhesion, the adhesive can be appropriately selected according to the polymer constituting the membrane or the material of the tube, and epoxy-based adhesives, silicone-based adhesives, acrylic-based adhesives, urethane-based adhesives, and the like can be used as the adhesive. For example, in a case where a tube containing a resin material having a melting point lower than that of the polymer material of the porous membrane is used, joining can be performed by adhesion.

<Application of Chamber for Transplantation>

The chamber for transplantation encloses the biological constituent and is used for transplantation of the biological constituent into the recipient. By using the chamber for transplantation, it is possible to prevent an immune rejection of the recipient with respect to the transplanted biological constituent. That is, the membrane for immunoisolation can be used for protecting biological constituents from an immune system of a recipient. In the present specification, a recipient means a living body to which transplantation is performed. A recipient is preferably a mammal, and is more preferably a human.

[Biological Constituent]

The biological constituent means a structure body derived from a living body. Examples of living bodies include viruses, bacteria, yeasts, fungal cells, insects, plants, mammals, and the like. It is preferable that a living body be generally a mammal. Examples of mammals include bovines, swine, sheep, cats, dogs, humans, and the like. The biological constituent is preferably a structure body derived from any of mammals.

Examples of biological constituents include organs, tissues, cells, and the like. Among these, cells are preferable as biological constituents. As cells, a single cell may be used or a plurality of cells may be used. It is preferable that a plurality of cells be used. A plurality of cells may be separated from each other or may be an aggregate.

The biological constituent may be obtained directly from a living body. In addition, particularly in a case where the biological constituent is a cell, the biological constituent may be directly obtained from a living body, or may be obtained by differentiation-induction of cells such as embryonic stem cells (ES cell), induced pluripotent stem cells (iPS cell), and mesenchymal stem cells. The cell may be a progenitor cell.

As a biological constituent, as one aspect, it is preferable to release a physiologically active substance. Examples of physiologically active substances include various hormones, various cytokines, various enzymes, and various other biologic factors in a living body. More specific examples include insulin, dopamine, factor VIII, and the like.

Here, insulin is a polypeptide (molecular weight of about 6000) in which an A chain of 21 amino acid residues and a B chain of 30 amino acid residues are linked via a disulfide bond. In insulin in a living body of a mammal is secreted from 13 cells in pancreatic islets of Langerhans. In a case of using insulin-secreting cells as the biological constituent in the present invention, insulin secreted may be human-type insulin or other mammalian-type (for example, porcine-type) insulin. Insulin may be insulin produced by a genetic recombination method. As a method for obtaining genetically modified insulin, for example, the description of Kadowaki Takashita: Diabetes Navigator (refer to 270-271, Takeo Tao, Yoshikazu Oka “Insulin Preparations of Present and Future,” Medical Review, 2002) can be referred to. Various types of insulin analogues (refer to, for example, H. C. Lee, J. W. Yoon, et al., Nature, 408, 483-488, 2000) may be used.

The biological constituent is preferably an insulin-secreting cell. Insulin-secreting cells are cells that can secrete insulin in response to changes in blood glucose level. The insulin-secreting cells are not particularly limited. Examples thereof include pancreatic (3 cells present in pancreatic islets of Langerhans. Pancreatic β cells may be human pancreatic β cells, or may be pancreatic β cells such as pigs and mice. For a method for extracting pancreatic β cells from a pig, reference can be made to the description in JP2007-195573A. In addition, the insulin-secreting cells may be cells derived from human stem cells (refer to, for example, Junichi Miyazaki, Regenerative Medicine, Vol. 1, No. 2, pp. 57-61, 2002), or cells derived from small intestinal epithelial stem cells (refer to, for example, Fumikomi Mineko et al., Regenerative Medicine, Volume 1, No. 2, pp. 63 to 68, 2002), or insulin-secretory cells into which a gene encoding insulin has been incorporated (refer to, for example, H. C. Lee, J. W. Yoon, et al., Nature, 408, pp. 483-488, 2000). Furthermore, the insulin-secreting cells may be pancreatic islets of Langerhans (refer to, for example, Horiyama, Kazumori Inoue, Regenerative Medicine, Volume 1, No. 2, pp. 69 to 77, 2002).

<<Device for Transplantation>>

The device for transplantation is a complex including at least a chamber for transplantation and a biological constituent. In the device for transplantation, the chamber for transplantation encloses the biological constituent therein.

In the device for transplantation, the chamber for transplantation may enclose only the biological constituent therein, or may enclose the biological constituent, and constituents or components other than the biological constituent therein. For example, the biological constituent may be enclosed in the chamber for transplantation together with a hydrogel, and preferably in a state of being enclosed in the hydrogel. In addition, the device for transplantation may contain pH buffers, inorganic salts, organic solvents, proteins such as albumin, or peptides.

The device for transplantation may contain only one biological constituent or may contain two or more biological constituents. For example, the device for transplantation may contain only a biological constituent which releases physiologically active substances for the purpose of transplantation, or which serves other functions of transplantation; or may further contain a biological constituent assisting functions of these biological constituents.

The device for transplantation may be, for example, a device to be transplanted intraperitoneally or subcutaneously. In addition, the device for transplantation may be a blood-vessel-connecting device. For example, in a case where insulin-secreting cells are used as the biological constituent, insulin secretion corresponding to a change in blood glucose level becomes possible by performing transplantation such that blood and the membrane for immunoisolation come into direct contact with each other.

Regarding the device for transplantation and chamber for transplantation, the description of Protein Nucleic Acid Enzyme, Vol. 45, pp. 2307 to 2312, (Okawara Hisako, 2000), JP2009-522269A, JP1994-507412A (JP-H06-507412A), and the like can be referred to.

### EXAMPLES

Characteristics of the present invention will be described in more detail with reference to the following examples and comparative examples. The materials, amounts used, proportions, treatment details, treatment procedures, and the like disclosed in the following Examples can be modified as appropriate as long as the gist of the present invention is maintained. Therefore, the scope of the present invention should not be limitedly interpreted by the specific examples described below.

<Production of Porous Membrane>

Polysulfone Porous Membrane

15 parts by mass of polysulfone (P3500 manufactured by Solvay), 15 parts by mass of polyvinylpyrrolidone (K-30), 1 part by mass of lithium chloride, and 2 parts by mass of water were dissolved in 67 parts by mass of N-methyl-2-pyrrolidone. Thereby, a stock solution for forming a membrane was obtained. This stock solution for forming a membrane was flow-cast on a surface of a PET film. The flow-cast membrane surface was exposed to air adjusted to 30° C. and relative humidity 80% RH, at 2 m/sec for 5 seconds. Immediately thereafter, the exposed membrane surface was immersed in a coagulation liquid tank filled with water at 65° C. The PET film was peeled off, and thereby a porous membrane was obtained. Thereafter, the immersed membrane surface was put into a diethylene glycol bath at 80° C. for 120 seconds, was thoroughly washed with pure water, and then was dried. Thereby, a porous membrane of Example 1 having a thickness of 50 pm was obtained.

In addition, by adjusting the thickness of the flow-cast stock solution for forming a membrane, an amount of water in the stock solution for forming a membrane (0.1 to 4 parts by mass), the relative humidity of temperature-controlled humid air after flow-cast (20 to 95% RH), and the temperature of the coagulation liquid tank (10° C. to 80° C.), membranes were produced in the same manner as Example 1 with controlling the bubble point diameter and the thickness of the porous membranes to the values shown in Table 1. Thereby, porous membranes of Examples 2 to 4 were obtained.

Cellulose Acetate Porous Membrane

5 parts by mass of cellulose acetate (CA1; degree of substitution 2.9) was dissolved in 55 parts by mass of dimethyl chloride, and 34 parts by mass of methanol was added to the solution little by little. Next, 0.2 parts by mass of glycerin and 6 parts by mass of pure water were added to the solution little by little to obtain a solution with almost no undissolved material, and the solution was filtered with a filter paper. Thereby, a dope was prepared.

The prepared dope was sent by a gear pump, was filtered, and then was flow-cast from a die on a polyethylene terephthalate (PET) film which was transported on an endless band.

The flow-cast membrane was dried with a drying air at 20° C. to 40° C. for 20 minutes. The film with PET was peeled off from the endless band, was dried with hot air at 80° C. to 120° C. for 15 minutes, and was wound with a winder. A number of fine pores were formed in the cellulose acetate on PET.

Cellulose acetate fine porous membrane was peeled off from PET using a peeling bar. Thereby, a porous membrane of Example 5 was obtained.

A porous membrane of Example 6 was obtained by producing a membrane in the same procedure as Example 4 except that cellulose acetate (degree of substitution 2.9) was replaced with another cellulose acetate (CA2; degree of substitution 2.5).

PVDF Porous Membrane

15 parts by mass of polyvinylidene fluoride resin, 65 parts by mass of dimethylacetamide, and 20 parts by mass of polyethylene glycol were mixed, and then 1 part by mass of polyoxyethylene sorbitan monooleate was added to the solution. Thereby, a mixed solution was obtained. The mixed solution was flow-cast on a glass plate, immediately immersed in water at 65° C. for 3 minutes, washed with water at 20° C., and then dried.

Subsequently, the membrane was immersed in 30 mass % of sodium hydroxide aqueous solution at 40° C. for 20 minutes, washed with water at 20° C., and then dried. Thereby, a porous membrane of Example 7 was obtained.

PC Porous Membrane

A polycarbonate film (thickness 20 μin) was irradiated with a helium ion beam such that an incidence angle was in a perpendicular direction to the main surface of the film. The irradiation density of argon ion was set to 1.0×10⁷ per 1 cm². The obtained film was immersed in 6N aqueous solution of sodium hydroxide at 60° C. for 2 minutes, and then was immersed in pure water at 60° C. for 10 minutes to be washed, and then was stored in a drying oven at 30° C. for 60 minutes to be dried. Thereby, a membrane of Example 8 was obtained.

PTFE Porous Membrane

A mixture of 100 parts by mass of PTFE powder and 20 parts by mass of liquid lubricant (liquid paraffin) was premolded and molded to have a round bar shape using a paste extrusion. The PTFE molding was rolled to have a thickness of 0.2 mm, the liquid lubricant was removed using an extraction solvent (decane), and then the extraction solvent was removed using a dryer heated to 150° C. Thereby, a PTFE sheet was obtained.

The obtained sheet was stretched approximately 5 times in the width direction (first stretching) and then stretched simultaneously in the biaxial direction (second stretching) using a biaxial stretching machine under the conditions of a stretching temperature of 300° C. and a stretching rate of 50%/sec. In the second stretching, the obtained sheet was stretched 7 times in each of two directions at a rate of 50%/sec. After stretching, the sheet was baked by heating at 380° C. for 10 minutes with the membrane dimensions fixed. Thereby, a membrane of Comparative Example 1 was obtained.

PET Porous Membrane

A biaxially stretched PET film having a desired heat shrinkage ratio was obtained by a method described in Example 1 of JP2011-208125A except that the heat fixation temperature was changed.

The film was irradiated with an argon ion beam such that an incidence angle was perpendicular to the main surface of the film. The irradiation density of argon ion was set to 2.0×10⁷ per 1 cm². The irradiated film was immersed in an etching treatment liquid (aqueous solution of 40 mass % of ethanol concentration and 14 mass % of potassium hydroxide concentration) at 60° C. for 1 minute. Thereafter, the film was taken out from the etching treatment liquid, was immersed in pure water at 60° C. for 10 minutes to wash, and then stored in a drying oven at 30° C. for 60 minutes to dry. Thereby, a membrane of Comparative Example 2 was obtained.

A membrane of Comparative Example 3 was obtained in the same procedure as that of Comparative Example 2 except that the heat fixation temperature was changed to those in the method described in Example 1 of JP2011-208125A.

<Evaluation of Porous Membrane>

Heat Shrinkage Evaluation

The produced membrane was cut into a circular shape with a diameter of 47 mm, and the center of the circle was marked. The cut membrane was immersed in hot water at 90° C. for 3 hours. The following A and B of the membrane were measured in a wet state at 25° C., and then the maximum heat shrinkage ratio, the minimum heat shrinkage ratio, and a difference of heat shrinkage ratio were obtained according to the following formulas.

A: Length of shortest straight line among straight lines passing through center mark (mm)

B: Length of longest straight line among straight lines passing through center mark (mm)

(Maximum heat shrinkage ratio)=(47−A)/47×100%

(Minimum heat shrinkage ratio)=(47−B)/47×100%

(Heat shrinkage ratio difference)=(Maximum heat shrinkage ratio)−(Minimum heat shrinkage ratio)

Bubble Point Diameter Evaluation

In a pore diameter distribution measurement test using a permporometer (CFE-1200AEX manufactured by SEIKA CORPORATION), a bubble point diameter of a membrane sample completely wetted by GALWICK (manufactured by PorousMaterials, Inc.) was evaluated after increasing an air pressure at 5 cm³/min.

<Production and Evaluation of Chamber for Transplantation (No Tube)>

Structural Evaluation

Two porous membranes were faced each other, and peripheral portions were directly joined to each other using an impulse heat sealer with a width of 1 mm. Thereby, a bag-shaped chamber for transplantation having a square shape of 20 mm (in which a non-joint portion of a porous membrane was a square shape of 20 mm) was produced.

The following (a) to (c) were evaluated after the chamber for transplantation was immersed in hot water at 90° C. for 3 hours.

(a) Warping/wrinkle evaluation (evaluation after drying)

1: No occurrence

2: Slight warping or wrinkles occurred

3: Small warping or wrinkles occurred

4: Large warping or wrinkles occurred

(b) Dimension evaluation (evaluation in wet state at 25° C.)

Evaluation was performed based on a shrinkage ratio of a side which was shrunk most among the four sides.

(c) Pore diameter change

A membrane was cut out from the chamber for transplantation, which was dried after being immersed in hot water, a bubble point diameter thereof was measured, and then a decrease “(before immersion−after immersion)/before immersion” with respect to a bubble point diameter before being immersed in hot water was calculated.

Transplantation Evaluation

Pancreatic islet cells were prepared using a pancreatic islets culture kit (rat) manufactured by Cosmo Bio.

Two porous membranes were faced each other, and peripheral portions (outer periphery) were directly joined to each other using an impulse heat sealer with a width of 1 mm while not joining a part thereof. Thereby, a bag-shaped chamber for transplantation having a square shape of 20 mm (in which a non-joint portion of a porous membrane was a square shape of 20 mm) was produced.

The chamber for transplantation was immersed in hot water at 90° for 3 hours and was dried.

The chamber for transplantation was sterilized with ethylene oxide gas, the pancreatic islet cells were inserted in the bag from unjoined portions, and then the chamber was sealed by joining the unjoined portions in the same manner described above.

The chamber for transplantation (device for transplantation) was transplanted into a model rat with type I diabetes, and an improvement rate (following formula) of blood glucose level after 2 weeks was evaluated.

(Improvement rate)=(blood glucose level before transplantation−blood glucose level after transplantation)/(blood glucose level before transplantation−normal level)×100%

<Production and Evaluation of Chamber for Transplantation with Tube>

Two porous membranes were faced each other, and a polyethylene tube (INTRAMEDIC Polyethylene Tubing PE100, manufactured by Becton, Dickinson and Company) was sandwiched between the central portions of one side of the peripheral portion. A stainless steel wire with a thickness of 1 2 mm was inserted into the tube, and using an impulse heat sealer, the porous membranes were directly joined to each other at the side into which the tube was sandwiched while joining the porous membrane and the tube. Subsequently, the remaining three sides were directly joined to each other in the same manner, and the steel wire inserted in the tube was removed. Thereby, a chamber for transplantation with the tube was produced.

Similarly to the chamber for transplantation (no tube), the obtained chamber for transplantation with the tube was immersed in hot water at 90° C. for 3 hours, and warping/wrinkle evaluation, dimension evaluation, and pore diameter change thereof were performed.

The obtained chamber for transplantation with the tube was sterilized with ethylene oxide gas, and pancreatic islet cells prepared in the same manner as described above were introduced into the chamber from the tube. Subsequently, a part of the tube was melted using the impulse heat sealer to close an inlet, and thereby the pancreatic islet cells were enclosed in the chamber. The obtained chamber for transplantation (device for transplantation) was transplanted into a model rat with type I diabetes, and an improvement rate (following formula) of blood glucose level after 2 weeks was evaluated.

(Improvement rate)=(blood glucose level before transplantation−blood glucose level after transplantation)/(blood glucose level before transplantation−normal level)×100%

Furthermore, the chamber was removed from the rat after two weeks from the transplantation, and an appearance of the chamber was observed to evaluate peeling of the tube joint portion after the transplantation.

The results are shown in Tables 1 to 3.

TABLE 1

| | | Membrane physical properties | | | | |
|---|---|---|---|---|---|---|
| | | Maximum heat | Minimum heat | Heat shrinkage difference | Thickness | Bubble point |
| | Polymer | shrinkage ratio | shrinkage ratio | (maximum − minimum) | μm | diameter μm |
| Example 1 | PSf | 0.21% | 0.21% | 0.00% | 50 | 1.5 |
| Example 2 | PSf | 0.21% | 0.21% | 0.00% | 35 | 1 |
| Example 3 | PSf | 0.21% | 0.21% | 0.00% | 40 | 0.3 |
| Example 4 | PSf | 0.03% | 0.02% | 0.01% | 80 | 0.5 |
| Example 5 | CA 1 | 1.49% | 1.17% | 0.32% | 75 | 0.65 |
| Example 6 | CA 2 | 4.68% | 3.40% | 1.28% | 75 | 0.65 |
| Example 7 | PVDF | 0.36% | 0.32% | 0.04% | 70 | 0.45 |
| Example 8 | PC | 0.30% | 0.26% | 0.04% | 30 | 0.05 |
| Comparative | PTFE | 14.89% | 10.43% | 4.47% | 24 | 0.45 |
| Example 1 | | | | | | |
| Comparative | PET | 5.32% | 3.19% | 2.13% | 50 | 1 |
| Example 2 | | | | | | |
| Comparative | PET | 4.89% | 3.30% | 1.60% | 33 | 1 |
| Example 3 | | | | | | |

PSf: Polysulfone;
CA: Cellulose acetate;
PVDF: Polyvinylidene fluoride;
PC: Polycarbonate;
PTFE; Polytetrafluoroethylene;
PET: Polyethylene terephthalate

TABLE 2

| | Evaluation of chamber for transplantation | | | |
|---|---|---|---|---|
| | Warping/ | Dimension | Pore diameter | Transplantation |
| | wrinkle | change | change | evaluation |
| Example 1 | 1 | 0.20% | 0.48% | 84% |
| Example 2 | 1 | 0.17% | 0.42% | 87% |
| Example 3 | 1 | 0.22% | 0.67% | 82% |
| Example 4 | 1 | 0.03% | 0.07% | 85% |
| Example 5 | 2 | 1.55% | 1.38% | 48% |
| Example 6 | 3 | 4.62% | 5.38% | 35% |
| Example 7 | 1 | 0.40% | 0.89% | 62% |
| Example 8 | 1 | 0.32% | 0.80% | 59% |
| Comparative | 4 | 14.66% | 24.44% | 7% |
| Example 1 | | | | |
| Comparative | 4 | 5.30% | 7.90% | 12% |
| Example 2 | | | | |
| Comparative | 4 | 4.91% | 7.30% | 18% |
| Example 3 | | | | |

TABLE 3

| | Evaluation of chamber for transplantation with tube | | | | |
|---|---|---|---|---|---|
| | | | | | Peeling of |
| | Warp- | Dimen- | Pore | Transplan- | tube joint |
| | ing/ | sion | diameter | tation | portion after |
| | wrinkle | change | change | evaluation | transplantation |
| Example 1 | 1 | 0.21% | 0.44% | 85% | N |
| Example 2 | 1 | 0.17% | 0.41% | 87% | N |
| Example 3 | 1 | 0.22% | 0.65% | 83% | N |
| Example 4 | 1 | 0.03% | 0.06% | 0% | Y |
| Example 5 | 2 | 1.54% | 1.37% | 48% | N |
| Example 6 | 3 | 4.66% | 5.40% | 36% | N |
| Example 7 | 1 | 0.38% | 0.88% | 61% | N |
| Example 8 | 1 | 0.33% | 0.78% | 59% | N |
| Comparative | 4 | 14.71% | 24.51% | 6% | N |
| Example 1 | | | | | |
| Comparative | 4 | 5.26% | 7.93% | 11% | N |
| Example 2 | | | | | |
| Comparative | 4 | 4.86% | 7.28% | 16% | N |
| Example 3 | | | | | |

What is claimed is:

1. A chamber for transplantation, comprising:
a membrane for immunoisolation at a boundary between an inside and an outside of the chamber for transplantation,
wherein the membrane for immunoisolation includes a porous membrane, and
in the porous membrane, a maximum heat shrinkage ratio among heat shrinkage ratios in a membrane surface direction after immersion in water at 90° C. for 3 hours is 0.00% to 5.00%, and a difference between the maximum heat shrinkage ratio and a minimum heat shrinkage ratio among the heat shrinkage ratios in the membrane surface direction is 0.00% to 1.30%.

2. The chamber for transplantation according to claim 1, further comprising:
a tube leading to the inside of the chamber for transplantation,
wherein a part of the porous membrane and a part of the tube are fusion welded, and
the maximum heat shrinkage ratio is 0.05% or more.

3. The chamber for transplantation according to claim 2, wherein the tube contains polyethylene, polyurethane, or polyvinyl chloride.

4. The chamber for transplantation according to claim 1, wherein the porous membrane contains a polymer.

5. The chamber for transplantation according to claim 1, wherein the membrane for immunoisolation is formed of a porous membrane containing a polymer.

6. The chamber for transplantation according to claim 1, wherein the porous membrane contains polysulfone or polyethersulfone.

7. The chamber for transplantation according to claim 1, wherein a thickness of the porous membrane is 25 μm to 100 μm.

8. The chamber for transplantation according to claim 1, wherein a bubble point diameter of the porous membrane is 0.02 μm to 25 μm.

9. A device for transplantation, comprising the chamber for transplantation according to claim 1 enclosing a biological constituent therein.

10. The device for transplantation according to claim 9, wherein the biological constituent releases a physiologically active substance.

11. The device for transplantation according to claim 10, wherein the physiologically active substance is insulin.

12. A method for transplanting a biological constituent into a recipient, comprising enclosing a biological constituent in the chamber for transplantation according to claim 1.

13. The method according to claim 12, wherein the biological constituent releases a physiologically active substance.

14. The method according to claim 13, wherein the physiologically active substance is insulin.

15. The method according to claim 12, wherein the biological constituent is insulin-secreting cells.

16. A method for preventing an immune rejection by a recipient in a case of transplantation, comprising enclosing a biological constituent in the chamber for transplantation according to claim 1.

17. The method according to claim 16, wherein the biological constituent releases a physiologically active substance.

18. The method according to claim 17, wherein the physiologically active substance is insulin.

19. A method for manufacturing a chamber for transplantation, using a membrane for immunoisolation,
wherein the membrane for immunoisolation includes a porous membrane, and
in the porous membrane, a maximum heat shrinkage ratio among heat shrinkage ratios in a membrane surface direction after immersion in water at 90° C. for 3 hours is 0.00% to 5.00%, and a difference between the maximum heat shrinkage ratio and a minimum heat shrinkage ratio among the heat shrinkage ratios in the membrane surface direction is 0.00% to 1.30%.

20. The method according to claim 19, comprising
providing the membrane for immunoisolation at at least part of a boundary between an inside and an outside of the chamber for transplantation.
